(19) United States
(12) Patent Application Publication
(10) Pub. No.: US 2026/0315522 A1
(43) Pub. Date: Oct. 15, 2026

(54) COMPOSITIONS AND METHODS FOR IMPROVING NITRIC OXIDE LEVELS IN INTRAORAL, NASAL AND/OR NASOPHARYNGEAL AREA

(71) Applicant: Shawn J. GREEN, Rockville, MD (US)

(72) Inventor: Shawn J. GREEN, Rockville, MD (US)

(21) Appl. No.: 19/107,256

(22) PCT Filed: Aug. 30, 2023
(86) PCT No.: PCT/US2023/073152

§ 371 Date: Feb. 27, 2025

Related U.S. Application Data
(60) Provisional application No. 63/402,149, filed on Aug. 30, 2022.

(51) Int. Cl. A61K 33/00; A23G 4/06; A23G 4/08; A23G 4/10; A23G 4/12; A61K 8/19; A61K 8/27; A61K 8/34; A61K 8/67; A61K 9/00; A61K 9/68; A61K 31/375; A61K 33/30; A61K 36/185; A61K 36/23; A61P 1/02; A61P 31/04; A61Q 11/00

(52) U.S. Cl. CPC A61K 33/00; A23G 4/064; A23G 4/068; A23G 4/08; A23G 4/10; A23G 4/126; A61K 8/19; A61K 8/27; A61K 8/347; A61K 8/676; A61K 9/0056; A61K 9/0058; A61K 31/375; A61K 33/30; A61K 36/185; A61K 36/23; A61P 1/02; A61P 31/04; A61Q 11/00; A61K 2800/22; A61K 2800/92

(57) ABSTRACT

The present disclosure relates to improving oral-nasal health by increasing, oral, intraoral and nasal cavity nitric oxide levels using a novel prebiotic nitrate-formulated chewing gum composition, together with an optional step comprising the use of saliva test strip to monitor pH, nitrate, and nitrite. The novel gum compositions and methods of the invention result in improved nitric oxide bioavailability and thereby increase antimicrobial nitric oxide levels beneficial for reducing dental caries, periodontitis, and nasopharyngitis, as well as reduce transmission of aerosolized viral particles acutely sensitive to nitric oxide, including SARS-COV.

### TECHNICAL FIELD

Embodiments of this disclosure relate generally to novel nitrate-based gum compositions useful for prolonging prebiotic nitrate exposure in the oral cavity of a subject resulting in a rapid and local increase in intraoral and nasopharynx nitric oxide gas. The embodiment also contributes to increasing systemic nitric oxide bioavailability.

### BACKGROUND OF THE INVENTION

Like many areas of the body, the mouth is teeming with bacteria, most of them harmless. Normally the body's natural defenses and good oral health care, such as daily brushing and flossing, can keep these bacteria under control. However, without proper oral hygiene, bacteria can reach levels that might lead to oral infections, such as tooth decay and gum disease. In addition, lifestyle activities, in particular smoking, or taking medications, such as decongestants, antihistamines, painkillers, antibiotics, and diuretics, or current oral hygiene practices, such as mouthwashes, can reduce saliva composition and flow as well as change the microbiome of the oral cavity, which in turn is detrimental to oral health. Saliva is important because it washes away food and neutralizes acids produced by bacteria in the mouth, helping to protect the mouth from microbial invasion or overgrowth that might lead to disease.

Oral health is essential to general health and quality of life. It is a state of being free from mouth and facial pain, oral and throat cancer, oral infection and sores, periodontal (gum) disease, tooth decay, tooth loss, and other diseases and disorders that limit an individual's capacity in biting, chewing, smiling, speaking, and psychosocial wellbeing. The most common oral diseases are dental cavities, periodontal (gum) disease, oral cancer, oral infectious diseases, trauma from injuries, and hereditary lesions.

Dental cavities are widely recognized as constituting serious concern with regard to oral health. Worldwide, 60-90% of school children and nearly 100% of adults have dental cavities, often leading to pain and discomfort. Tooth decay (cavities) is one of the most common chronic conditions of childhood in the United States. Untreated tooth decay can cause pain and infections that may lead to problems with eating, speaking, playing, and learning. In the United States, about 1 of 5 (20%) children aged 5 to 11 years have at least one untreated decayed tooth; and about 1 of 7 (13%) adolescents aged 12 to 19 years have at least one untreated decayed tooth. The percentage of children and adolescents aged 5 to 19 years with untreated tooth decay is twice as high for those from low-income families (25%) compared with children from higher-income households (11%). There are threats to oral health across the lifespan. Nearly one-third of all adults in the United States have untreated tooth decay.

Periodontitis is a set of inflammatory diseases affecting the periodontium, i.e., the tissues that surround and support the teeth. Periodontitis involves progressive loss of the alveolar bone around the teeth, and, if left untreated, can lead to the loosening and subsequent loss of teeth. Periodontitis is caused by microorganisms that adhere to and grow on the tooth's surfaces, along with an overly aggressive immune response against these microorganisms. Periodontitis manifests as painful, red, swollen gums, with abundant plaque. Symptoms may include redness or bleeding of gums while brushing teeth, using dental floss, or biting into hard food (e.g. apples); recurrent swelling of the gum; halitosis and a persistent metallic taste in the mouth; gingival recession resulting in apparent lengthening of teeth; deep pockets between the teeth and the gums (pockets are sites where the attachment has been gradually destroyed by collagenases); and loose teeth. Periodontitis also has been shown to have effects outside of the mouth. For example, periodontitis has been linked to increased inflammation as indicated by increased levels of C-reactive protein and Interleukin-6. In addition, periodontitis has been shown to increase the risk for a number of other diseases, including but not limited to, stroke, myocardial infarction, atherosclerosis, diabetes, and pre-term labor. Severe periodontal (gum) disease, which may result in tooth loss, is found in 15-20% of middle-aged (35-44 years) adults. In addition, dental cavities and periodontal disease are major causes of tooth loss. Complete loss of natural teeth is widespread and particularly affects older people. Globally, about 30% of people aged 65-74 have no natural teeth.

Oral cancer constitutes another concern in oral health. The incidence of oral cancer ranges from one to 10 cases per 100,000 people in most countries. The prevalence of oral cancer is relatively higher in men, in older people, and among people of low education and low income. Tobacco and alcohol are major causal factor and contribute to disruption of the microbiome, specifically, nitrate reducing bacteria on the tongue. As such smoking reduces the health promoting microbiome on the tongue.

Oral health might affect, be affected by, or contribute to various diseases and conditions, including: endocarditis, an infection of the inner lining of the heart (endocardium), and typically occurs when bacteria or other germs from another part of the body, such as the mouth, spread through the bloodstream and attach to damaged areas in the heart; cardiovascular disease, research suggests that heart disease, clogged arteries and stroke might be linked to the inflammation and infections that oral bacteria can cause; pregnancy and birth, periodontitis has been linked to premature birth and low birth weight; diabetes, since diabetes reduces the body's resistance to infection the gums are put to at risk and gum disease appears to be more frequent and severe among people who have diabetes (research shows that people who have gum disease have a harder time controlling their blood sugar levels); HIV/AIDS, oral problems, such as painful mucosal lesions, are common in people who have HIV/AIDS in addition, almost half (40-50%) of people who are HIV-positive have oral fungal, bacterial or viral infections; osteoporosis, since this condition causes bones to become weak and brittle it might be linked with periodontal bone loss and tooth loss; Alzheimer's disease, tooth loss before age 35 might be a risk factor for Alzheimer's disease; other conditions that might be linked to oral health include Sjogren's syndrome an immune system disorder that causes dry mouth and eating disorders. Asymptomatic transmission of SARS-CoV-2 remains a concern and owing to the peripheral anatomical location and frequent exposure of oral tissues to the external environment. With that said, saliva plays a major role in the asymptomatic spread of SARS-CoV-2, hence, saliva actively participates in SARS-CoV-2 transmission.

The burden of oral diseases and other chronic diseases can be decreased simultaneously by addressing common risk factors. These include: decreasing sugar intake and maintaining a well-balanced nutritional intake to prevent tooth decay and premature tooth loss; consuming fruit and vegetables that can protect against oral cancer; stopping tobacco use and decreasing alcohol consumption to reduce the risk of oral cancers, periodontal disease and tooth loss; using protective sports and motor vehicle equipment to reduce the risk of facial injuries; safe physical environments, and most importantly, ensuring proper oral hygiene. Dental cavities can be prevented by maintaining a constant low level of fluoride in the oral cavity, however, fluoride and chlorhexidine mouthwashes may also lower the healthy microbiome in the oral that contributes to antimicrobial nitric oxide.

Similar to the oral cavity, the nasopharynx is a primary site of colonization by respiratory pathogens and it constitutes a port of entrance to the respiratory tract. The nasopharynx often harbors bacterial and viral pathogens responsible for both middle ear and sinus infections. The most common condition that affects the nasopharynx is nasopharyngitis, otherwise known as the common cold. This swelling of the nasal passages and throat is sometimes called an upper respiratory infection, or rhinitis. In nasopharyngitis, a virus, often rhinovirus, infects the nasopharynx. The nose is the primary entry site and target of SARS-CoV-2. Asymptomatic transmission of SARS-CoV-2 remains a concern owing to the peripheral anatomical location and frequent exposure of both the oral and nasopharynx tissues to the external environment. Therefore, the oral-nasopharynx plays a critical role in the asymptomatic spread of SARS-CoV-2, hence, saliva and exhaled aerosolize water-mucus droplets from the nose participates in SARS-CoV-2 transmission.

### Salivary Nitrate and Oral Health

Nitrite, derived from nitrate through nitrate reducing bacteria on the tongue surface, is considered cytocidal and cytostatic to common oral pathogens involved in caries and in periodontal disease, especially when acidified. Therefore, an increase in nitrate secretion and a subsequent increase in salivary nitrite may contribute to the overall protective effect against those infections conditions, affecting both hard and soft oral tissues. It is known that salivary glands may respond to periodontitis by enhancing the protective potential of saliva. Thus, it is likely that the increment in salivary nitrate-nitrite concentration, in patients with periodontal disease, may be due to an increase in nitrate secretion as a response of salivary glands to the inflammatory process. In accordance with this hypothesis, it has been reported that patients with oral candidiasis have increased salivary nitrates and nitrites concentration.

High salivary nitrate and high nitrate-reducing capacity in the oral cavity was found to be protective against dental caries in a study of 209 children. Salivary nitrate and nitrite levels, counts of Streptococcus mutans and Lactobacillus spp., and caries experience were recorded, and compared with control subjects, a significant reduction in caries and counts of Streptococcus mutans and Lactobacillus spp. was found in patients with high salivary nitrate. Production of nitrite from salivary nitrate by commensal nitrate-reducing bacteria likely limit the growth of cariogenic bacteria because of the production of antimicrobial oxides of nitrogen, including nitric oxide. (J. J. Doel, M. P. Hector, C. V. Amirtham, L. A. Al-Anzan, N. Benjamin, R. P. Allaker, Protective effect of salivary nitrate and microbial reductase activity against caries, Eur. J. Oral Sci. 112 (2004) 424-428.)

Others have shown that the antimicrobial agent nitric oxide is formed in the mouth and its concentration is directly related to salivary nitrite, which in turn is related in part to dietary nitrate intake. Here the investigators showed that nitrite, via the bioconversion from nitrate, under acidic conditions had an inhibitory effect, through NO production, on Streptococcus mutans, Lactobacillus casei and Actinomyces naeslundii. Whereas the growth of S. mutans was inhibited by a more acid pH, the addition of nitrite caused a marked, further dose-dependent reduction in bacterial numbers after 24 hours of exposure. Similar effects were observed with A. naeslundii and L. casei. The ability of these bacteria to recover from nitrite exposure was also markedly affected by nitrite concentration. At acidity levels below pH 7, low concentrations of nitrite (0.2 mM) caused effective complete killing of S. mutans, with similar effects on the other organisms tested. These results demonstrate that nitrite in saliva blocks cariogenic bacteria. (L. S. Silva Mendez, R. P. Allaker, J. M. Hardie, N. Benjamin, Antimicrobial effect of acidified nitrite on cariogenic bacteria, Oral Microbiol. Immunol. 14 (1999) 391-392, C. E. Radcliffe, R. Lamb, A. S. Blinkhorn, D. B. Drucker, Effect of sodium nitrite and ascorbic acid on the growth and acid production of Streptococcus mutans, J. Dent. 31 (2003) 367-370.)

Consistent with other reports, nitrite derived from nitrate under acidic conditions inhibited the growth of periodontal disease pathogens Fusobacterium nucleatum, Eikenella corrodens and Porphyromonas gingivalis. (P. Allaker, L. S. Silva Mendez, J. M. Hardie, N. Benjamin, Antimicrobial effect of acidified nitrite on periodontal bacteria, Oral Microbiol. Immunol. 16 (2001) 253-256.) Sanchez et al (2014) reports that an increase in nitrate likely contributes to the overall protective effect against periodontal-associated pathogens affecting both hard and soft oral tissues. They suggest that an increase in salivary nitrate-nitrite concentration, in patients with periodontal disease, is due to an increase in nitrate secretion as a response of salivary glands to the inflammatory process. In accordance with this hypothesis, it has been reported that patients with oral candidiasis have increased salivary nitrates and nitrites concentration. In both cases, it is suggested that the elevation of nitrate in the oral cavity during advance disease is an immune response to such infection. (Total salivary nitrates and nitrites in oral health and periodontal disease. Sánchez G A, Miozza V A, Delgado A, Busch L.Nitric Oxide. 2014 Jan. 30; 36:31-5). Numerous additional studies have highlighted the benefits of salivary nitrate levels: Li et al (2007) suggest that elevated salivary nitrate reduces oral acidity hence protects against tooth decay. (Oral Microbiol Immunol. 2007 February; 22 (1): 67-71. Salivary nitrate—an ecological factor in reducing oral acidity. Li H1, Thompson I, Carter P, Whiteley A, Bailey M, Leifert C, Killham K.) Along similar lines, Radcliffe (2002) suggests that exogenous nitrite acidified by metabolic products of acidogenic bacteria in the mouth will be converted to products which inhibit growth of Streptococcus mutans. (Effects of nitrite and nitrate on the growth and acidogenicity of Streptococcus mutans. Radcliffe C E, Akram N C, Hurrell F, Drucker D B. J Dent. 2002 September-November; 30(7-8):325-31.) In addition to susceptible acidified nitrite derived from nitrate, including cariogenic bacteria Streptococcus mutans and various periodontal bacteria, Fusobacterium and P. gingivalis, the sulfate-reducing bacteria, Desulfovibrio spp., which is considered an etiologic agent associated with chronic periodontitis and implicated to sulfate odors is directly inhibited by both nitrite and nitrate. Here, growth is inhibited with 0.2 mM nitrate which is consistent with other observations. Salivary nitrate and nitrite may also have antimicrobial effects on Desulfovibrio species. (Mitsui T, Fujihara M, Harasawa R. Biosci Biotechnol Biochem. 2013; 77(12):2489)

Nitrate concentrations are elevated in body with diet nitrate-rich plants, such as leafy greens and beetroots, and certain oral bacteria convert nitrate into nitrite and the human body can effectively convert nitrite into nitric oxide by certain enzymatic and non-enzymatic processes, resulting in beneficial outcomes, including, reducing blood pressure and inhibiting the spread of disease-causing pathogenic organisms.

The dietary pathway or the L-arginine-independent, nitrate-nitrite-nitric oxide dietary pathway, coined, enterosalivary nitrate pathway, is when dietary nitrate is swallowed and absorbed in the proximal gastrointestinal tract and via the circulatory system is absorbed and concentrated in the salivary gland where it is secreted into the mouth and subsequently reduced by nitrate-reducing bacteria to nitrite after 30 to 90 minutes; nitrite is subsequently swallowed and is further reduced to nitric oxide and related intermediates in the stomach, blood stream, and tissue.

### Oral Nitrate-Reducing Microbiome in Oral-Nasopharynx Cavities and Enterosalivary Nitrate Pathway for Oral and Upper Respiratory Health

As mentioned previously certain oral bacteria, specifically, nitrate-reducing bacterial convert the nitrate into nitrite and the human body can effectively convert nitrite into nitric oxide by several enzymatic and non-enzymatic processes, however, this is dependent upon the enterosalivary nitrate pathway.

Different research groups have focused on the systemic benefits of dietary nitrate with the intent to shift the oral microflora and this is usually with ingested dietary source, hence, dependent on the enterosalivary nitrate pathway as opposed to increasing the ‘hang-time’ of nitrate inside the mouth and assessing the intraoral nitric oxide gas itself. There is no current teaching or availability of resources that enable directly administrating a nitrate-based formula to increase nitrate-reducing and nitrite-reducing activity to generate nitric oxide within the mouth and nasopharynx that is independent of the enterosalivary nitrate pathway resulting in the immediate enhancement of intraoral and nasal cavity nitric at doses well below the Acceptable Daily Intake (ADI) as well as extend the duration of local and systemic nitric bioavailability over several hours.

Current studies focus on nitrate-reducing capabilities in the oral cavity but at very high levels and beyond the ADI involving the enterosalivary nitrate pathway. Burgleigh et al. (2019) observed that after 7 days of beetroot consumption, there is an increase in the salivary pH and Neissera, and a decrease in Prevotella, Actinomyces and Streptococcus. In this study, multiple high doses of nitrate had to be ingested at levels far above the ADI.

In similar fashion others have shown a beneficial impact of dietary nitrate in shifting the oral microbiome through ingestion of nitrate sources, including Velmurugan et al. (2016) showing that after 6 weeks of daily nitrate-rich beetroot juice consumption, containing 372 mg per serving nitrate-reducing species, Rothia mucilaginosa and Neisseria flavescens, increased. Vanhatalo et al., (2018) reported on changes in oral microbiota detected in saliva after nitrate supplementation ingestion showing an increase in nitrate-reducing bacteria, Rothia and Neisseria and a decrease in disease associated, Prevotella and Veillonella. Jockel-Schneider et al., (2016) reports on a reduction of gingival inflammation in patients after 14 days of nitrate intake.

Mira and Rosier, et al. (2018, 2022) suggest nitrate for use in reducing or preventing oral dysbiosis and increasing oral eubiosis, by changing the bacterial composition of oral biofilms with effects before 24 hours of a biofilm-mediated oral disease and thereby increases the concentration of nitrate in the saliva of the mouth. Furthermore, they claim either topical or ingested, hence, as to the later, the dependence of the enterosalivary loop and in either case suggest an increase concentration of nitrate in the saliva of the mouth with a composition that is either nitrate-rich vegetable extract which is a beetroot extract, an antioxidant and/or a nitrate-reductase enzyme cofactor which is molybdenum, a salt thereof or a molybdenum-rich vegetable extract. Similar to many others, the ingested formula may indeed increase the saliva nitrate levels after traveling through the circuitous loop from the mouth to the stomach and back to the saliva gland and mouth via the circulatory system. With that said, Rosier and Mira (2022) also claim to achieve a shift in the nitrate-reducing microbiome with topical administration through toothpaste, mouthwash, oral gel, food extract and chewing gum with a nitrate dose of 3 μg with a 0.1 mM, which is a hypothetical construct that has not been reduced to practice nor have shown intraoral or nasopharynx increase in nitric oxide production immediately or persistently within human subjects.

Rosier et al (2022), Prebiotic and probiotic treatment to reduce oral dysbiosis and promote eubiosis, WO2021122741A2, report that nitrate levels in saliva collected in the morning in a healthy donor under fasting conditions, after intake of a nitrate-rich supplement (220 mg nitrate in 200 ml water) right after 0 hours resulting in two peaks, hence, illustrating the direct or immediate increase of nitrate due to the topical supplement contact (0.5 h) and the indirect increase due to the salivary gland activity that recycles nitrate from plasma (2.5 h), however, they failed to detect intraoral or nasopharynx nitric oxide and the concentration to achieve this transient bi-phasic response below the ADI level. Furthermore, they failed to differentiate that chewing gum comparable to other local nitrate source (provided as a toothpaste or rinse) results in different outcomes. Further, although Rosier et al. claim that chewing gum, toothpaste and rinse can be equally effective in increasing local and systemic levels to achieve an antimicrobial outcome in the mouth and nasal cavity they fail to demonstrate, reduce to practice, or provide any evidence to validate such a claim.

Rosier et al (2020, WO2021122741A2) among others provide nitrate formulated solutions with the addition of specific nitrate-reducing bacterial in the presence of nitrate, including Bryan's (2022, WO2014182632A1). Neither Rosier or Bryan provide any evidence or suggestion related to the health implications of increasing intraoral-nasal nitric oxide production. Both Rosier and Bryan rely on the enterosalivary pathway and pay no consideration that such a pathway can be circumvented through the use of specially formulated chewing gum.

Within the nasopharynx (to include paranasal sinuses and nasal cavities) as noted by Lundberg et al (1999) in multiple papers, nitric oxide is produced by the innate immune response when exposed to bacterial and viral infections. Nitric oxide possesses antimicrobial activity against broad range of bacteria, parasites, fungi and viruses.

Nitric oxide delivered in an exogenous gaseous form, might easily enter the pulmonary milieu and be useful in optimizing the treatment of uncontrolled pulmonary disease with specific actions directed at reducing bacterial burden, reducing inflammation and improving clinical symptoms, especially, when it is well understood that nasal nitric oxide levels are markedly reduced in patients who generally have mucus filled paranasal sinuses and obstructed sinus ostia, Kartagener's syndrome, cystic fibrosis, acute sinusitis as review by Lundberg et al (1999).

In children with Kartagener's syndrome, consisting of sinusitis, bronchiectasis, and situs inversus, nasal nitric oxide levels are extremely low compared with healthy, age matched controls (Lundberg et al., 1999). In like fashion, smoking was associated with decreased exhaled nitric oxide. The greatest smoking-related declines in exhaled nitric oxide occurred in older subjects. This suggests that smoking is associated with age-related declines in exhaled nitric oxide.

Nasal nitric oxide levels are also very low in patients with cystic fibrosis. Baraldi et al measured nasal nitric oxide in a group of children with acute sinusitis and found low nasal NO levels and chronic sinusitis is associated with more than a 50% reduction in nasal NO levels. In general, nasal nitric oxide levels are lower in subjects with sinus disorders.

Av-Gay et al. (2013), (US20200276229A1), claims that some respiratory disorders and physiological conditions can be treated by inhalation of gaseous nitric oxide delivered from a mechanical device or canister of nitric oxide or by an external spray device delivering acidified nitrite.

The use of nitric oxide by inhalation can prevent, reverse, or limit the progression of disorders such as acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery, acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Inhaled nitric oxide can also be used to treat cystic fibrosis, chronic pulmonary hypertension, bronchopulmonary dysplasia, chronic pulmonary thromboembolism and idiopathic or primary pulmonary hypertension or chronic hypoxia.

As pointed out by Av-Gay et al. (2013), nitric oxide has a half-life in the body of less than several seconds and a radius of action of approximately 200 microns from its site of origin, beyond which it is inactivated through binding to sulfhydryl groups of cellular thiols or by nitrosylation of the heme moieties of hemoglobin to form methemoglobin (MetHb). MetHb reductase reduces nitric oxide to nitrates in the blood serum. Nitrate has been identified as the predominant nitric oxide metabolite excreted in the urine, accounting for more than 70% of the nitric oxide dose inhaled. Nitrate is cleared from the plasma by the kidney at rates approaching the rate of glomerular filtration. Blood levels of MetHb in healthy humans are typically less than 2%. Potential side effects of high dose nitric oxide treatment hence include the binding of nitric oxide to hemoglobin and the formation of MetHb, which could lead to decreased oxygen transport, and the capacity of NO to act as a nitrosylating agent on proteins and other cell constituents. Formation of MetHb and increased levels thereof have been observed in previous studies of gaseous nitric oxide inhalation by healthy human individuals, wherein inhalation of gaseous nitric oxide at 128 ppm for 3 hours and at 512 ppm for 55 minutes has been reported to drive the levels of MetHb over the safe threshold of 5% [Borgese N. et al., J. Clin. Invest., 1987, 80, 1296-1302; Young J. D. et al., Intensive Care Med., 1994, 20, 581-4 and Young J. D. et al., Brit. J. Anaesthesia, 1996, 76, 652-656].

Inhaled nitric oxide from an external device or canisters, albeit demonstrated to be beneficial under healthcare monitoring, comes with its risks especially if consider on a daily use with little if any professional healthcare oversight. Lundberg, a leading authority of respiratory nitric oxide biology raises concerns regarding the potential use of nitric oxide as a therapeutic agent in various scenarios. Lundberg et al., (Eur. Respir J, 1994), Primarily nasal origin of exhaled nitric oxide and absence in Kartagener's syndrome, state that almost all nitric oxide in exhaled air originates from the upper airways whereas in orally and nasally exhaled air the same individuals showed considerably higher nasal nitric oxide concentrations. Most of the nitric oxide from the upper airways is, thus, likely to be produced in the nasal cavity, since exhaled nitric oxide levels were also higher during nasal compared to oral breathing in all subjects, including healthy controls. Furthermore, direct nasal sampling yielded very high nitric oxide values and nitric oxide from the upper airways will follow the airstream with every inhalation and thus, continuously flush the lower airways. As cited by Lundberg et al. (1994), inhaled nitric oxide at concentrations as low as 100 ppb significantly decrease pulmonary vascular resistance in patients with pulmonary hypertension, indicating that nasally derived nitric oxide could be physiologically important in the lung and, thus, act as an airborne, or “aerocrine” factor. The author goes on to indicate that nasally produced nitric oxide gas is bacteriostatic and exhibits antiviral properties, hence, participate in the unspecific primary mucosal defense line against infections.

In summary, despite advances in the understanding of nitric oxide and its effect on physiology, there remains a need for efficient, targeted, prolonged and sustained delivery of nitric oxide compositions for improving oral and -nasopharynx health. What is also needed are compositions and methods that bypass the enterosalivary nitrate circuit, and enable subjects to intermittently and rapidly augment intraoral and nasal nitric oxide. Additionally, what is needed are systems such as those utilizing rapid saliva test strips for monitoring pH and nitric oxide metabolites to ensure continuous salivary nitrate, nitrite, and nitric oxide bioavailability, so that subjects can enhance and maintain elevated nitric oxide levels with the periodic or intermittent chewing of nitrate-formulated compositions multiple times per day while remaining below the current acceptable daily intake.

### SUMMARY OF THE INVENTION

In an embodiment, the present disclosure relates to the use of a nitrate-based formulation delivered in a compression-type chewing gum to prolong prebiotic nitrate exposure in the oral cavity. Use of the chewing gum by a subject enables a rapid and local increase in intraoral and nasopharynx nitric oxide gas. The chewing gum of the present disclosure also contributes to improved systemic nitric oxide bioavailability resulting from the subsequent swallowing of nitrate and bioconversion via the enterosalivary nitrate-nitrite-nitric oxide pathway. In an embodiment, the chewing gum composition is designed to enable nitric oxide levels to be below the acceptable daily intake of 3.7 milligrams per kilogram of body weight per day (mg/kg bw/day). The design and composition of the gum allows for intermittent administration of the nitrate-based chewing gum throughout the day to immediately and locally increase oral-nasopharynx nitric oxide production and prolong the delayed systemic nitric oxide bioavailability. In contrast to customarily available nitrate-based supplements such as mouthwash rinses, toothpaste or ingestibles, the novel gum compositions claimed herein uniquely enable subjects to achieve optimal nitric oxide levels both locally and systemically.

In an embodiment, as validated and determined by salivary nitrite measurements, the chewing gum compositions of the present disclosure uniquely contribute to a bi-phasic nitric oxide response: first by providing an instantaneous local increase in the oral and nasal cavity; and second as a response after swallowing, wherein followed by absorption in the gut, nitrate is concentrated in the salivary gland as a result of enterosalivary circulation.

In an aspect, the invention further utilizes information about nitric oxide levels and acidity-alkalinity in an individual's oral cavity, specifically, monitoring real-time pH and nitric oxide metabolites, nitrate and nitrite, with rapid self-testing saliva test strips to make adjustment as to the dosing of the nitrate-formulated chewing gum.

Not wishing to be bound by the following theory, the gum composition of the present disclosure is thought to act by shifting the oral microbiome to a higher abundance of the nitric oxide promoting bacteria with the corresponding increase the in ratio of salivary nitrite-to-nitrate (>1) and an increase in the pH (>7). This shift in the oral microbiome corresponds to a decrease in bacteria associated with disease related to pathogens that contribute to gum inflammation, tooth decay, halitosis, and cardiometabolic complications.

Provided herein are novel unanticipated approaches for improving oral hygiene and nasopharynx health by rapidly elevating intraoral and/or nasopharynx nitric oxide levels, optionally combined with a means to monitor levels with an affordable, easy to use, rapid self-testing method to monitor three critical endpoints: pH, nitrate, and nitrite.

In an embodiment, the disclosure herein comprises a novel chewing gum composition that rapidly increases intraoral-nasal cavity nitric oxide levels thereby bypassing the L-arginine-independent, nitrate-nitrite-nitric oxide dietary pathway. In contrast, currently available nitric oxide supplement products, as well as prior art and publications, are exclusively based on improving nitric oxide levels by depending on the L-arginine-independent, nitrate-nitrite-nitric oxide dietary pathway.

In an embodiment, the invention provides a methodology for optimizing oral-nasal health by coupling or bundling a novel nitrate-formulated chewing gum with a saliva self-test to gain real-time information concerning the levels of salivary nitric oxide analytes in the oral cavity. This information may be utilized to maximize the antimicrobial activity of such analytes and consequently improve oral, nasopharyngeal and sinus health with intermittent chewing of nitrate-formulated gum. More particularly, rapid saliva testing that may be used at-will and directly in the oral cavity for monitoring nitric oxide status enables regular monitoring and can be incorporated into a routine for improving oral and nasopharynx health.

In an embodiment, the present disclosure provides real-time feedback through the detection of saliva analytes and a biomarker of nitric oxide, to enables individuals to make real-time adjustments to oral hygiene regimens and to optimize oral health. According to the present disclosure, users can rapidly, in a real-time fashion, evaluate nitric oxide levels in their oral cavities, and determine a corrective course for improving and maintaining oral hygiene.

In an embodiment, the invention provides a novel programmatic approach to oral hygiene comprising a nitrate-formulated chewing gum to extend nitrate exposure in the oral cavity to enhance or shift the microbiome from an acid or cariogenic microflora, to a nitrate-reducing microbiome.

By increasing the ‘hang-time’ of nitrate exposure, the nitrate-reducing oral microbiome and areas within the mouth of acidic or low pH, such as inflammatory gingival sulcus surrounding teeth and teeth coated with acidic cariogenic biofilms, nitrite bioconversion to nitric oxide is facilitated. Unlike toothpaste and rinses, the chewing gum composition claimed herein provides a unique advantage by both prolonging exposure of the oral cavity to nitrate, while stimulating salivary secretion to contribution to downstream nitrate-nitrite-nitric oxide pathway with the subsequent ingestion of nitrate provided by the gum (which would not be the case for toothpaste or mouth rinse).

In an embodiment, the invention comprises the use of a nitric oxide generating chewing gum used in conjunction with intra-daily self-monitoring with a saliva test strip for promoting consumer compliance behavior. Colorimetric test strip outcomes may be recorded and tracked on electronic devices such as wearables or mobile phones providing reminders and updates to both user and dentist or healthcare provider through wireless messaging capabilities as to compliance and adherence to oral healthcare.

In an embodiment, the present disclosure provides systematic approaches for improving oral and sinus health. Nitric oxide decreases inflammation and pathogenic microbes and accordingly, use of the novel gum compositions as disclosed herein enables the decrease in the incidence of health issues related to oral health and the upper respiratory system, including but not limited to, dental cavities, tooth decay, gum disease, periodontitis, oral cancer and viral infections or virus sequestered in saliva and sinus, including nasopharyngitis. The gum compositions also decrease the viral load of nitric oxide-sensitive coronavirus, including SARS CoV-1 and SARS-CoV-2.

These and other aspects, features and advantages of the present disclosure will become apparent after a review of the following detailed description of the disclosed embodiments and the appended claims.

### BRIEF DESCRIPTION OF FIGURES

The present disclosure is best understood from the following detailed description when read in conjunction with the accompanying drawings. It is emphasized that, according to common practice, the various features of the drawings are not necessarily to scale. On the contrary, the dimensions of the various features are arbitrarily expanded or reduced for clarity. Like reference numerals denote like features throughout specification and drawings.

FIG. 1 provides a schematic summary of nitric oxide bioavailability comparison of a nitrate-based chewing gum (thick line) versus an equal amount of nitrate-derived from an ingested capsule or beverage (thin line) as measured with MYFITSTRIP® saliva test strips for nitrite, the surrogate marker for nitric oxide, over the course of time.

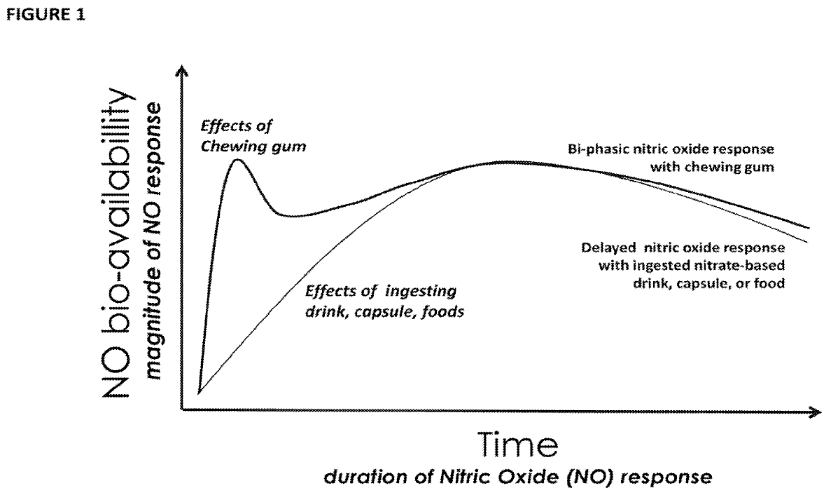

FIG. 2 provides a graph showing intraoral nitric oxide gas formation (A), saliva nitrate (B), and saliva nitrite (C) at various times after chewing two (2) plant-derived, nitrate-formulated gum for 5 mins at 0 minutes and 45 minutes and then discarded. ARROW(S) represents the time point of administering chewing gum for 5 min and the number of ARROW(S) indicates the number of pieces.

FIG. 3 provides a graph showing intraoral nitric oxide gas formation (A), saliva nitrate (B), and saliva nitrite (C) at various times after chewing one (1) potassium nitrate-formulated gum for 5 minutes at 0 minutes and then discarded. ARROW(S) represents the time point of administering chewing gum for 5 min and the number of ARROW(S) indicates the number of pieces.

FIG. 4 provides a graph showing intraoral nitric oxide gas formation (A), saliva nitrate (B), and saliva nitrite (C) at various times after chewing three (3) potassium nitrate-formulated gum for 5 minutes at 0 minutes for 5 minutes and then discarded. ARROW(S) represents the time point of administering chewing gum for 5 min and the number of ARROW(S) indicates the number of pieces.

FIG. 5 provides a graph showing intraoral nitric oxide gas formation (A), saliva nitrate (B), and saliva nitrite (C) after chewing potassium nitrate-formulated gum at two different points for 5 minutes. In this example, 1 piece is administered at 0 minutes for a duration of 5 min and another at 150 minutes for 5 minutes and each discarded. ARROW(S) represents the time point of administering chewing gum for 5 min and the number of ARROW(S) indicates the number of pieces.

FIG. 6 provides a graph showing intraoral nitric oxide gas (A) and nasal exhaled nitric oxide at various times after chewing potassium nitrate-formulated gum. In this example, 1 piece is administered at 0 minutes for a duration of 5 min discarded.

FIG. 7 shows the relative abundance of the bacterial species before and after chewing potassium nitrate-formulated gum. Whole genome metagenomic sequencing or Shotgun sequencing was used to look at all the DNA present in a microbiome sample. Saliva samples were in subjects with low nitrite-to-nitrate ratio with Probiotic Nitric Oxide Tests by MyFitStrip® and low saliva pH (below 7) as detected with Oral Health Strips by MyFitStrips®. Saliva was collected according to the methodology provided by Bristle Oral Health Labs. Upon collection, subject chewed potassium-ascorbic acid formulated gum for for 5-10 min twice over a period 3-4 hours. At that time, nitrite-to-nitrate and pH were measure and a second saliva sample was collected for Shotgum sequencing analysis according to the procedures of Bristle Labs. In this example, improving oral and nasopharynx health by enhancing nitric bioavailability by increasing the relative abundance of Rothia aeria, Rothia mucilaginosa, Neisseria flavescens, Neisseria subflava, Haemophilus parainfluenzae within saliva after 6 hrs from chewing 3 pieces at 0, 2 and 4 hrs at of said gum during this period with corresponding reduction of disease associated bacteria Tannerella forsythia, Treponema socranskii, Fusobacterium periodonticum, Porphyromonas gingivalis, Streptococcus constellatus, Fusobacterium nucleatum, Parvimonas micra, Prevotella melaninogenica, Prevotella histicola, Candida albicans.

### DETAILED DESCRIPTION

The following detailed description is exemplary and explanatory and is intended to provide further explanation of the present disclosure described herein. Other advantages, and novel features will be readily apparent to those skilled in the art from the following detailed description of the present disclosure. Texts and references mentioned herein are incorporated in their entirety, including U.S. Provisional Patent Application No. 63/402,149 filed on Aug. 30, 2022.

For purposes of the description hereinafter, it is to be understood that the embodiments described below may assume alternative variations and embodiments. It is also to be understood that the specific articles, compositions, and/or processes described herein are exemplary and should not be considered as limiting.

In 1998, the Nobel Prize for medicine was awarded to Robert F. Furchgott, Louis J. Ignarro and Ferid Murad for discovering the importance of nitric oxide in the cardiovascular system. These scientists demonstrated that nitric oxide, is a short-lived, endogenously produced gas that acts as a signaling molecule in the body. Signal transmission by a gas, produced by one cell, which penetrates membranes and regulates the function of other cells was recognized for the first time as an entirely new principle for signaling in the human organism. Related research proved the crucial role that nitric oxide plays in such fundamental biological processes as regulation of blood pressure, functioning and malfunctioning of the immune system, and activation of mechanisms in the central nervous system affecting everything from gastric motility to memory to behavior.

The present disclosure is based upon the discovery that nitric oxide has antimicrobial activity in the oral cavity: more specifically, the inventors herein have observed that oxides of nitrogen produced non-enzymatically and enzymatically from the serial chemical reduction of nitrate to nitrogen oxides, in particular nitric oxide, are potently antimicrobial. To optimize and direct the antimicrobial activity of nitric oxide, the inventors have recognized the need to deliver nitric oxide locally as well as systemically. Further the need to create, sustain and monitor a threshold level of the appropriate precursor within the oral cavity is accomplished by the use of salivary test strips. This disclosure provides a teaching for compositions and that enable the optimization of nitrate metabolites to maintain a healthy and antimicrobial environment in the oral and nasal cavity of a subject. This goal may be accomplished by the use of chewing gum to slowly release a nitrate-formula to increase the exposure of the critical oral microbiome to the nitrate, which behaves as a prebiotic to both shift the cariogenic and acid promoting bacteria to a health beneficial nitrate-reducing bacterium.

For products such as toothpaste or mouth rinse made with a similar formula, the ‘hang-time’ of the nitrate within the oral cavity is limited to a few seconds, or possibly one or two minutes. In contrast, the nitrate-based gum composition as claimed herein provides a longer and more sustained level of nitrate in the oral cavity. When the nitrate-based gum of the invention is chewed for five minutes, followed by intermittent chewing throughout the day, nitrate levels in the oral cavity are established such that oral health is improved, caries and decay causing microorganisms (such as ‘bad’ bacteria) are diminished and friendly oral microflora are able to flourish. The use of the nitrate-based gum compositions is particularly advantageous as the act of chewing gum is more practical than brushing teeth with a nitrate-based toothpaste or using a nitrate based mouthwash several times a time (to reach equivalent levels of nitrate in the oral and/or nasopharyngeal cavity).

An additional consequence of using nitrate-based gum, is that the nitrate is subsequently swallowed which extends nitric oxide bioavailability when nitrate is recycled through the enterosalivary pathway. The enterosalivary nitrate pathway is the way in which many nitric oxide supplement and nitric oxide-potent foods, rich in inorganic nitrate, are metabolized to generate nitric oxide. Accordingly, the nitrate-based chewing gum of the present disclosure (or a slow-release lozenge similarly formulated) is uniquely suited for immediate local and delayed systemic nitric oxide formation and delivery. Yet another advantageous feature of the gum compositions claimed herein is that nitric oxide gas is generated immediately, which would not be the case with the swallowing of a nitrate capsule or nitrate-rich drink or food. Again, chewing gum delivery provides a unique advantage resulting in a bi-phasic nitric oxide curve observed within minutes and subsequently 90 min later, hence. The local delivery in the mouth directly enhances the antimicrobial activity within the oral and nasal cavity. The systemic delivery that happens as a result of the enterosalivary nitrate pathway “loops back” and further improves the nitric oxide environment of the oral cavity. In addition, the elevated nitric oxide levels also benefit the subject has a whole, with particular regard to cardiovascular health.

Nitrate itself is an innocuous precursor, which only produces antimicrobial species when converted to nitrite, and subjected to acid conditions. Lactobacilli sp. transiently produces sufficient acid in the mouth after a carbohydrate meal to control the growth of oral pathogens, but a moderate intake of nitrate is a desirable prerequisite since it also contributes to increasing pH conditions as described by Rosier et al (2018). In addition to having antimicrobial activity, nitrite and nitrogen oxide in the oral cavity also has an antiviral effect. Though not wishing to be bound by the following theory, the inventors herein have found that viruses, as opposed to bacteria for example, react differently to a nitrogen oxide complex: namely, when exposed to a nitrogen oxide complex, comprising for example nitrate and/or acidified nitrite, while the complex may effect replication to a degree, more importantly it modifies the virally infected cells such that the immune system can better recognize the viral particles.

In the case of coronavirus disease (COVID-19), the inventor previously established that restoring nitric oxide improves endothelial dysfunction and contributes to pulmonary vasodilation, antithrombotic, and direct antiviral activity (Microbes and Infection, Volume 22, Issues 4-5, 2020, p.149-150). Nitric oxide interferes with the interaction between coronavirus viral S-protein and its cognate host receptor, ACE-2. Nitric oxide-mediated S-nitrosylation of viral cysteine proteases and host serine proteases, TMPRSS2, which are both critical in viral cellular entry, appear to be nitric oxide sensitive. Based on a report of improved lung function during the 2003 SARS outbreak, FDA's emergency expanded use of nitric oxide gas is now underway for treating COVID-19.

Alternatively, dietary inorganic nitrate has been shown in multiple studies to be effective at restoring endothelial function, reducing pulmonary and arterial hypertension, and promoting antimicrobial activity. As described previously, it is well understood that dietary inorganic nitrate is bio-converted to nitric oxide through a series of well-defined steps beginning with the friendly microflora on the tongue reducing nitrate to nitrite, which is subsequently reduced to nitric oxide in the gut, blood stream, and various organs, including the lung. The formation of inorganic nitrite and S-nitrosothiols is absorbed into the circulation where it acts as a transitory storage pool for subsequent nitric oxide production. The conversion of inorganic nitrite to nitric oxide is expedited in conditions of acidosis or hypoxemia which occurs in regions of the pulmonary vasculature in lungs of chronic obstructive pulmonary disease (COPD) patients and those that exhibit acute respiratory distress syndrome as observed in coronavirus infected lungs. Reportedly, consumption of inorganic nitrate for 8 days in COPD populations increased lung nitric oxide by 200% and reduced respiratory symptoms. Restoring nitric oxide through dietary inorganic nitrate may be a consideration for prevention and early treatment which would operate at two-levels: reverse platelet-endothelial dysfunction and associated thrombosis as well as lower viral burden and if generated locally in the oral and nasal cavity, this invention enables a reduction in COVID transmission.

The effect of salivary nitrate excretion is to provide a precursor for the generation of nitrogen oxides by the chemical reduction of the nitrite. In the mouth, bacteria rapidly reduce nitrates to nitrites (1). Nitrite is further reduced in the sulcus to antimicrobial nitrogen oxide species, including nitric oxide (1-3). The acid conditions of the sulcus are further elevated and protonate nitrite to form nitrous acid (1-4). The nitrous acid in turn dissociates to form oxides of nitrogen as shown below.

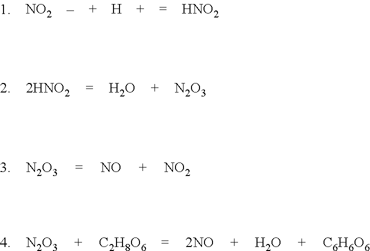

Endogenous and dietary nitrate is actively concentrated by salivary glands to more than ten times the concentration in plasma and secreted in saliva. The nitrate-based gum composition of this disclosure provides sources of nitrate as an immediate precursor for the serial reduction in the mouth for antimicrobial activity in the mouth and nasal cavity. In certain embodiments, antimicrobial effects are pronounced if periodontal disease with acid producing bacterial in the sulcus is present.

Oral conversion of nitrate to nitrite is rapid and generally takes place on the surface of the tongue in mammals by commensal nitrate-reducing bacteria (mainly to the posterior third of the tongue) and can be subsequently reduced to nitrogen oxide, including nitric oxide, in the sulcus of infected pockets in the presence of low pH or acidic environments. Alternatively, as disclosed herein, nitrate and ascorbic acid can further accelerate the chemical reduction or enhance the formation of antimicrobial nitric oxide in the oral and nasal cavity. In an embodiment of the present invention, a moderately acid is provided. Provided herein is a novel oral hygiene regimen comprising the use of a source of nitrate for serial reduction in the mouth for the direct treatment and/or prevention of oral and upper respiratory infections, coupled with the monitoring of oral nitric oxide level via nitrite and nitrate levels as a biomarker for antimicrobial activity with the intent to increase adherence to daily oral health practice, for example, intermittent chewing of nitrate formulated gum.

The present disclosure meets the unmet medical and health needs of improving and monitoring oral-nasal health. To reduce the number of caries-producing organisms in dental plaque and prevent the advancement of gingivitis and periodontal disease, the present disclosure provides a novel programmatic approach comprising the use of a nitrate-based components, in conjunction with a saliva test strip to monitor the presence of nitrogen oxides after brushing. Additionally, this programmatic approach extends beyond anti-caries and can be used to treat and prevent infection with C. albicans or other harmful organisms of the oral cavity that are susceptible to nitrogen oxides. With the increase in nasal nitric oxide gas, the benefits are extended to the upper respiratory area, including the nasal cavity and sinus, which are susceptible to viral infection and serves as reservoir of such infection for transmission, such as SARS-CoV-1 and SARS-CoV-2.

The present disclosure provides a novel source of nitrate in the form of a nitrate rich gum. In an embodiment, the nitrate-rich gum as described herein may be composed of potassium nitrate or a plant powder source derived from spinach, kale, arugula, celery, beets among other leafy greens that are rich in nitrate. The nitrate salt or plant derived nitrate powder source may be mixed with a gum base, which can be, but not limited to a natural sources, such as chicle, and non-natural sources, including, but not limited to, butadiene-styrene rubber, paraffin, and various waxes used in making chewing gum, In certain embodiments, the gum may further comprise sugar-free sweeteners, such as xylitol, with the addition of both natural and artificial flavors, including spearmint, orange, among other herb and plant sources, such as fennel, and fruit flavors.

In one aspect, the present invention comprises a method for improving oral-nasal antimicrobial activity health in a subject comprising, use of chewing gum consisting of 2 to 8% potassium nitrate (by weight based on the total weight of the gum) and may comprise a ratio of 2:1 to 1:4 ascorbic acid. In an embodiment, the invention comprises a novel approach for the treatment and/or prevention of bacterial, viral, or fungal conditions in the oral cavity and a programmatic approach to monitor it for maximal antimicrobial activity. Though not wishing to be bound by the following theory, it is expected that acidification of the nitric oxide precursors occurs in the sulcus or subgingival space of infected pockets and said source of nitrite ions derived from nitrate precursor are further reduced to form antimicrobial nitrogen oxide, in particular nitric oxide. An aspect of the disclosure pertains to the source of nitrate, optionally reduced by microflora in the oral cavity that can be further reduced in the presence of salicylic acid and/or ascorbic acid which increases the antimicrobial effects within the oral cavity. A further aspect of the disclosure comprises the use of saliva test strips to monitor nitrate wherein to achieve antimicrobial activity, test strips are used to monitor the nitrite in the oral cavity.

Suitable strips for use with the present disclosure comprise a single device, having a three-step method for collecting, transferring, and measuring saliva fluid analytes, specifically for nitrogen oxide anions, nitrate, nitrite, analytes of and biomarkers for nitric oxide as well as pH. In an embodiment, saliva strips, such as the strips that are commercially available as MYFITSTRIP® (Rockville, MD, USA), not only monitor nitric oxide and pH status, but they also provide an instant readout such that the user can make informed decisions, in real-time, about maintaining a level sufficient of nitrite in the oral cavity to maintain oral health. The MYFITSTRIP® saliva strips enable users (such as those prone to dental problems) who need a sensitive, easy to use, and affordable test that can be used 3-4 times daily to make hygiene and dietary adjustments as needed to maintain optimal levels of nitric oxide.

The unique design of strip used herein enables easy collection of the test fluid, without requiring an additional vessel, or direct use of fingers to collect or distribute the fluid. The MYFITSTRIP® saliva strips overcome the shortcomings of other nitric oxide test strips such as BERKELEY TEST® and HUMANN INDICATOR TEST STRIPS which are limited to nitrite detection, whereas MYFITSTRIP® saliva strips are designed to detect nitrite, nitrate, and pH. MYFITSTRIP® saliva strips prevent false-negative results unlike the others which means that when the other test strips are negative the subject would not know if the oral microbiome for bioconversion is present or whether the supplement, gum, drink, or foods was nitrate-rich.

In an embodiment, provided herein are chewing gum compositions comprising a gum base combined with: (a) potassium nitrate and ascorbic acid; or (b) potassium nitrate, ascorbic acid and polyphenol extract; or (c) potassium nitrate, ascorbic acid and zinc; or (d) potassium nitrate, ascorbic acid, zinc, and polyphenol extract; or (e) plant-derived inorganic nitrate and ascorbic acid; or (f) plant-derived inorganic nitrate, ascorbic acid, and zinc; or (g) plant-derived inorganic nitrate, ascorbic acid, zinc, and polyphenol extract. The chewing gum compositions may further comprise sweeteners, a gum base or bulk filler, flavorings, lubricants, flow agents or combinations thereof. The chewing gum compositions of the invention may be utilized to enhancing nitric oxide levels in the oral, intraoral and/or nasal cavity of a subject.

The gum base may comprise chicle, gelatin, pectin, beeswax, paraffine waxes, rosin, butyl rubber, polyvinyl acetates, microcrystalline cellulose, plant fibers, or a combination thereof. The gum composition may be formed into a stick, or a tablet, or the gum may have a liquid center.

In certain embodiments, the sweeteners may comprise sugars, non-sugars, maltitol, saccharin, aspartame, sorbitol, sucralose, isomalate, erythritol, xylitol, plant-derived sweeteners, stevia, agave, coconut sugar, honey, monk fruit extract, sugar blends, or blends of sugar alcohols, and/or combinations thereof.

In certain embodiments, the chewing gum composition of the disclosure further comprise flavorings, the flavorings may comprise natural or artificial flavorings or a combination thereof. Such flavorings may comprise mint, garden mint, mixed mint, spearmint, peppermint, wintergreen, fruit, watermelon, strawberry, blackberry, raspberry, orange, lemon, lime, clementine, tangerine, grapefruit, mango, yuzu, banana, apple, peach, pear, plum, pineapple, pomegranate, ginger, cinnamon, menthol, or chocolate.

In certain embodiments, the potassium nitrate comprises 1-5% of the gum composition. The potassium nitrate may be derived from a plant source, wherein the plant source is selected from the group consisting of leafy greens, comprising celery, beet, arugula, Swiss chard, beetroot and/or combinations thereof.

In certain embodiments, the ascorbic acid comprises plant-derived ascorbic acid, including but not limited to, acerola-derived ascorbic acid; the gum compositions may comprise 0.5-10, 7-15% wt/wt ascorbic acid, in a ratio of nitrate-to-ascorbic acid of 2:1 to 1:4.

In certain embodiments, the gum composition comprises 1-10% zinc, 4-12% L-arginine, 1-5% N-acetylcysteine, or polyphenol fruit-derived extract at 2-10 mg/dose of activated anthocyanins (per g extract at 10:1) or combinations thereof.

In certain embodiments, the gum composition comprises 1% zinc, 6-8% L-arginine, 1-5% N-acetylcysteine, or polyphenol fruit-derive extract at 2-10 mg/dose of activated anthocyanins (per g extract at 10:1), or a combination thereof.

In an embodiment, the amount of nitrate per chewing gum composition is 0.15 to 2.5 mM.

In certain embodiments, a subject may establish and maintain nitric oxide levels in the oral and/or intraoral-nasal cavity, and optionally also increase total body bioavailability of nitric oxide by chewing a first gum composition for at least 3-7 minutes, discarding it, and then chewing additional gum compositions intermittently throughout a 12 hour period (chewing subsequent gum compositions for 3-7 minutes). Each gum composition chewing period may be separated by 1-30, 30-60, 60-90, 90-120, 120-150 minute increments.

In an embodiment, enhancing nitric oxide levels in the oral, intraoral and/or nasal cavity of a subject comprises increasing the relative abundance of beneficial microflora, wherein beneficial oral microflora includes but is not limited to one or more of, Rothia aeria, Rothia mucilaginosa, Neisseria flavescens, Neisseria subflava, Haemophilus parainfluenzae. In an embodiment, a relative increase in beneficial oral microflora comprises approximately 1% or more compared to before chewing the gum composition after a minimum of 4-6 hours.

In an embodiment, enhancing nitric oxide levels in the oral, intraoral and/or nasal cavity of a subject comprises decreasing the relative abundance of harmful oral microflora, wherein harmful oral microflora includes, but is not limited to one or more of, Tannerella forsythia, Treponema socranskii, Fusobacterium periodonticum, Porphyromonas gingivalis, Streptococcus constellatus, Fusobacterium nucleatum, Parvimonas micra, Prevotella melaninogenica, Prevotella histicola, Candida albicans, and diseases associated therewith. In an embodiment, a relative decrease in harmful oral microflora comprises approximately 1% or more compared to before chewing the gum composition after a minimum of 4-6 hrs.

In an embodiment, enhancing nitric oxide levels in the oral, intraoral and/or nasal cavity of a subject comprises and improvement of oral health comprising a reduction in dental plaque, dental cavities, tooth decay, gingivitis, halitosis, bacterial infections, fungal infections, viral infections, nasopharyngitis and associated-sinus and oral infections.

In certain embodiments, the post-fasting salivary concentration of nitrate and nitrite during chewing and post 5 minutes is at least 100 mg/L or more and 10 mg/L or more, respectively, as assessed by saliva test strips for nitrate and nitrite.

In certain embodiments, chewing the gum composition as claimed herein results in an increase in saliva pH to 6.5-8.0; wherein the increase in salivary pH results in a reduction of tooth demineralization, oral acidosis, and/or acidogenic bacteria.

In an embodiment, the gum compositions of the disclosure comprise potassium nitrate ascorbate-zinc, gum base, sorbitol, maltitol, xylitol, isomaltose, natural flavors, potassium nitrate, magnesium stearate, acerola, sucralose, silicon dioxide, and zinc citrate.

In an embodiment, the gum compositions of the disclosure comprise plant-based acerola, gum base, sorbitol, maltitol, xylitol, isomaltose, celery extract, natural flavors, magnesium stearate, acerola, sucralose, silicon dioxide, polyphenols and glycerin.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the term “subject” should be construed to include subjects, for example medical or surgical subjects, such as humans and other animals requiring supplemental or therapeutic intervention.

In the present disclosure the singular forms “a,” “an,” and “the” include the plural reference, and reference to a particular numerical value includes at least that particular value, unless the context clearly indicates otherwise. Thus, for example, a reference to “a bead” or “a nano structure” is a reference to one or more of such structures and equivalents thereof known to those skilled in the art, and so forth. When values are expressed as approximations, by use of the antecedent “about,” it will be understood that the particular value forms another embodiment. As used herein, “about X” (where X is a numerical value) preferably refers to +10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such a listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms but is generally insufficient to cause adverse side effects. The specific effective amount for any subject will depend upon a variety of factors including the age, body weight, general health, sex and diet of the patient; the time of administration; the route of administration; and like factors well known in the field of health.

Disclosed are the components to be used to prepare a composition of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of ingredients A, B, and C are disclosed as well as a class of ingredients D, E, and F and an example of a combination substance, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

As used herein, gum means a soft, chewable, cohesive substance designed to be chewed without being swallowed. In an embodiment, the gum may be composed of gum base, sweeteners, softeners/plasticizers, flavors, colors, and, optionally a hard or powdered polyol coating. Its texture may be reminiscent of rubber because of the physical-chemical properties of its polymer, plasticizer, and resin components, which contribute to its elastic-plastic, sticky, chewy characteristics.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

The following specific examples will illustrate the invention as it applies to the methods of improving oral health by detecting and monitoring biomarkers such as pH nitrate, and nitrites in saliva. It will be appreciated that other examples, including minor variations in procedures will be apparent to those skilled in the art, and that the invention is not limited to these specific illustrated Examples.

### EXAMPLES

The chewing gum formulas used in Examples 1-7 are generally summarized as shown below:

Formula 1: Potassium nitrate-ascorbate-zinc

| Ingredient | % Formula |
|---|---|
| Gum base | 24.96% |
| Sorbitol | 19.97% |
| Maltitol | 14.98% |
| Xylitol | 12.98% |
| Isomaltose | 9.98% |
| Natural flavors | 6.57% |
| Potassium nitrate | 5.19% |
| Magnesium stearate | 3.09% |
| Acerola | 1.10% |
| Sucralose or Stevia | 0.72% |
| Silicon dioxide | 0.40% |
| Zinc citrate | 0.06% |
| Total | 100.00% |

Formula 2: Plant-base nitrate-acerola

| Ingredient | % Formula |
|---|---|
| Gum base | 25.03% |
| Sorbitol | 19.64% |
| Maltitol | 12.76% |
| Xylitol | 11.78% |
| Isomaltose | 9.82% |
| Celery Extract | 7.85% |
| Natural Flavors | 7.23% |
| Magnesium stearate | 3.04% |
| Acerola | 1.18% |
| Sucralose or Stevia | 0.79% |
| Silicon dioxide | 0.39% |
| Polyphenols | 0.39% |
| Glycerin | 0.10% |
| Total | 100.00% |

### Example 1

Bioconversion of Plant-Derived Nitrate in the Oral Cavity to Nitrite and Subsequent Reduction to Nitric Oxide Gas with a Corresponding Elevation of pH

As discussed herein, dietary inorganic nitrate and its reduced forms nitrite and nitric oxide, respectively, are of critical importance for host defense in the oral cavity. High concentrations of salivary nitrate are linked to a lower prevalence of caries due to growth inhibition of cariogenic bacteria and the increase in pH. Anti-cariogenic activity is dependent upon the bioconversion of nitrate to nitrite and downstream reactive nitrogen oxides, specifically, antimicrobial nitric oxide.

This example demonstrates that bioconversion of nitrate to nitrite to the local production of nitric oxide gas takes place within the mouth, immediately. Although it is well established that reactive nitrogen oxides exhibit antimicrobial activity, at present no method or invention ensures the validation of the nitrate and nitrite source and whether nitrate is converted immediately and locally within the oral cavity.

NObreath® (NOB; Bedfont, Kent, UK) was used to measure nitric oxide gas and MyFitStrip® (Rockville, MD USA) was used to measure salivary pH and salivary nitric oxide metabolites, nitrate and nitrite.

NOB was used to measure nitric oxide gas. These devices were assessed based on the National Institute for Health and Clinical Excellence (NICE) guideline. NOB is designed to measure fractional exhaled nitric oxide (FeNO) in the human breath and a normal FeNO test is any number less than 25 parts per billion (ppb) in adults. Levels above this value have been used to assess inflammatory response in the lung where inducible nitric oxide synthase is activated in immune cells in response to an inflammatory response. NOB is used to evaluate nitric oxide gas generated locally in the mouth.

Participants were screened in advance, excluding asthmatics, individuals with respiratory infections and healthy individuals with levels about 35 ppb which was due to nitrate-rich beet or leafy green diets or excessive nasal breathing within 6-18 hour prior to testing. All included healthy subjects consistently exhaled between 10-30 ppb which was influenced with exercise and diet, especially, nitrate-rich plant-based foods and diets and excessive nasal breathing.

A total of 4-6 subjects were evaluated 2-4 time over the course of 2 weeks. The Figures are measurements from a single individual and representative of the other 4-6 subjects. Participates underwent the measurement in a sitting position, always holding the monitor upright. After inhaling to total lung capacity, participants then exhaled through the mouthpiece, ensuring that the visual indicator marker was held in the middle of the bands for proper measurement. The exhalation time was approximately 10 seconds. All participants were asked to repeat the measurement 3 times to verify the repeatability of the device. The baseline values ranging between 10-35 ppb is reflective of lung and respiratory tract sourced nitric oxide prior to chewing gum.

MyFitStrip® was used to measure salivary pH and salivary nitrate and nitrite. After participates completed the breath test with NOB, MyFitStrip® collection pad was place on the tongue to collect saliva for 5 seconds, removed from the mouth and fold over to contact the test pad end and held together for 10 second to ensure saliva transfer to colorimetric test pad. After 10 seconds, the folded strip was release showing the color pad which was compared to a color scale of a pH range of 4.5, 5.5, 6.5, 7.0, 7.5, 9.0, a nitrate range consisting of 0, 5, 10, 25, 50, 75, 100 mg/L, and a nitrite range consisting of 0, 0.5, 2, 5, 10, 25, 50 mg/L. MyFitStrip® test pad is based on the well-established Griess reagent reaction for both nitrate and nitrite colorimetric detection and measurement.

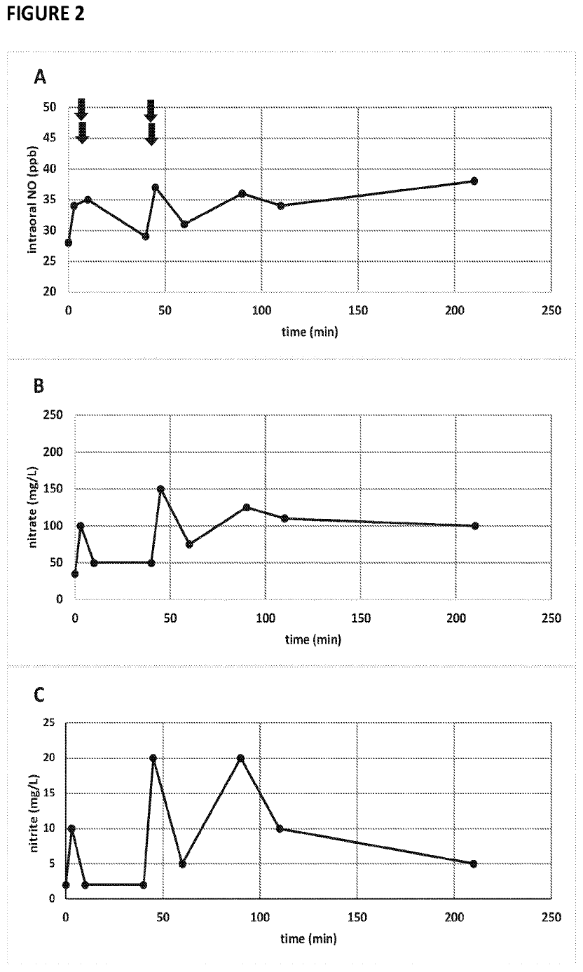

Example 1 (FIG. 2) shows both an immediate increase antimicrobial nitric oxide gas (A) with the increase in the corresponding precursor and metabolite, nitrate (B) and nitrite (C), respectively, with the chewing of two plant-derived nitrate gum pieces for 5 minutes beginning at time 0 and again at 45 min and then discarded. ARROW(S) represents the time point of administering chewing gum for 5 min and the number of ARROW(S) indicates the number of pieces. Methods for detecting nitric oxide gas and salivary metabolites are described above.

The present example also monitored pH which in all cases the subjected the saliva pH prior to chewing gum ranged between 5.5 to 6.5 and immediate increase to 7.0 to 7.5 within 3-5 min of chewing gum.

Nitrate rich dietary source have been shown to exhibit a protective effect against caries by an increase of salivary pH (Sustaining elevated levels nitrite in the oral cavity through consumption of nitrate-rich beetroot juice in young healthy adults reduces salivary pH, Hohensinn, B., et al. (2016) Nitric Oxide 60:10-15). With the bioconversion of nitrate to nitrite, the pH of saliva rose from 7.0 to 7.5, confirming the anti-cariogenic effect of provide a dietary NO3 source. These results clearly indicate that nitrate-rich dietary source significantly prevents the acidification of the salivary pH if consumed regularly and bioconversion of NO3 to NO2 occurs as determined by a saliva NO test strip. However, Hohensinn et al (2016), showed that dietary NO3 needs to be constantly ingested in order to benefit from the antimicrobial effects and other biological functions of nitric oxide. Hence, the importance of enterosalivary nitrate-nitrite-nitric oxide loop whereas an immediate pH shift was observed upon chewing within minutes.

It would not be unreasonable to predict that if oral bioconversion levels were sustained throughout the day, the mouth would remain alkaline, as reflected by a cytocidal effect of the acid-forming bacteria. In this case, the strips would be used to remind the subject to intermittently chew nitrate-based gum and/or slow-release lozenge especially when levels of detected nitrite begin to naturally fall through the day. In doing so, salivary pH values would seldom become acidic and harmful to the teeth.

pH is a prominent indicator for the presence and number of cariogenic bacteria, and elevated pH values result from a decline in the number of acid-producing bacteria. Thus, to shift from a transient cytostatic to a lasting cytocidal effect on cariogenic bacteria, chewing nitrate-based gum incorporated into one's oral health program as regulated by intra-daily readings with MYFITSTRIP® test strip for pH, nitrate, and nitrite. Individuals will vary as to how long their bioconversion of nitrate-nitrite-nitric oxide gas, locally and systemically, hence, it will be critical to monitor frequently with test strips and, if levels are falling, replenish with nitrate-gum to increase a local oral source before acidogenic bacteria reestablish.

This invention demonstrates that nitrate-rich source holds potential effects against dental caries via bioconversion of nitrate to nitrite to nitric oxide resulting in elevating pH levels which is predictably a reduction of acidogenic cariogenic bacteria. However, persistent anti-cariogenic effect which is likely due to the clearance of nitrate which needs to be periodically replenished based on test strip outcomes.

The current example demonstrates an immediate production of nitric oxide gas (A) with a corresponding reduction of nitrite (C) from nitrate (B) within 3 to 5 min of chewing gum. A subsequent chewing of gum at 45 min resulted in a similar response as the 3-5 min timepoint although at a slightly elevated and persistent elevation of gas, nitrate and nitrite that persisted for a minimum of an additional hour. The intermittent chewing was found to be additive and persistent in elevating antimicrobial levels of nitric oxide in the oral cavity and as such it resulted in maintaining and improving oral hygiene: 1. dietary nitrate source in a delivery format that optimizes immediate local levels of nitrate, including but not limited to nitrate-rich gum or slow releasing gummy or chewy or lozenge and the like and 2. a saliva test strips to both ensure, indirectly, nitrate content and validate bioconversion of nitrate to antimicrobial nitrite.

### Examples 2-4

Bioconversion of Potassium Nitrate-Ascorbate-Zinc Chewing Gum in the Oral Cavity to Nitrite and Subsequent Reduction to Nitric Oxide Gas with a Corresponding Elevation of pH

Wherein the methods, experiment protocol, and measurements for salivary pH, nitrate, nitrite, and nitric oxide gas is described above for plant-derived gum is also followed for the potassium-ascorbate-zinc chewing gum.

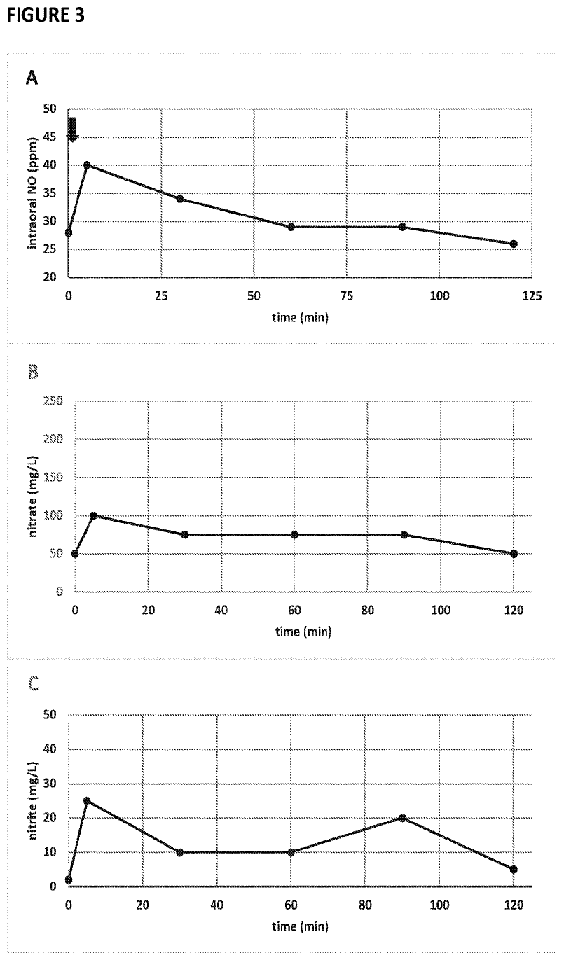

Example 2 (FIG. 3) shows both an immediate increase in antimicrobial nitric oxide gas (A) with the increase in the corresponding precursor and metabolite, nitrate (B) and nitrite (C), respectively, with the chewing of one potassium nitrate-based chewing gum piece for 5 minutes and then discarded. ARROW(S) represents the time point of administering chewing gum for 5 min of chewing. Methods for detecting nitric oxide gas and salivary metabolites are described above. Example 2 demonstrates an immediate production of nitric oxide gas (A) with a corresponding reduction of nitrite (C) from nitrate (B) within 3 to 5 min of chewing gum and that persisted for a minimum of an additional hour with antimicrobial nitric oxide levels with an associated pH of >7.5 during this time period.

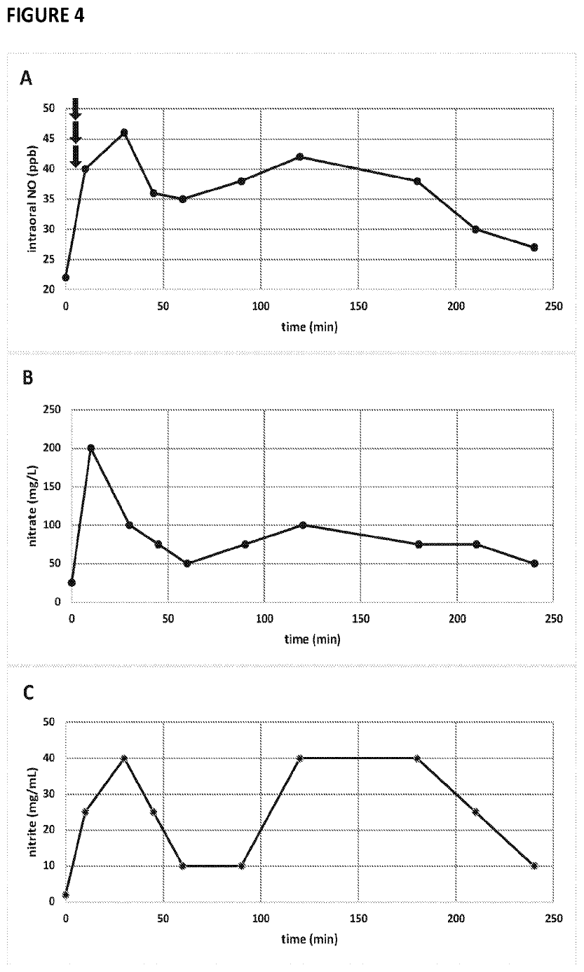

Example 3 (FIG. 4) shows an immediate and sustain elevation of intraoral nitric oxide gas formation (A), saliva nitrate (B), and saliva nitrite (C) at various times after chewing three (3) potassium nitrate-formulated gum for 5 minutes at 0 minutes and then discarded. ARROW(S) represents the time point of administering chewing gum for 5 min and the number of ARROW(S) indicates the number of pieces. Example 3 demonstrates an immediate and sustained production of nitric oxide for 3 hours with an associated pH of >7.5 during this time period. The bi-phasic nature of dosing is best represented by this example where intraoral is immediate and sustained for 30-60 min which allow adequate time for the ingested nitrate to recycle back to the mouth to sustain nitric oxide in the mouth for an additional 2-3 hours.

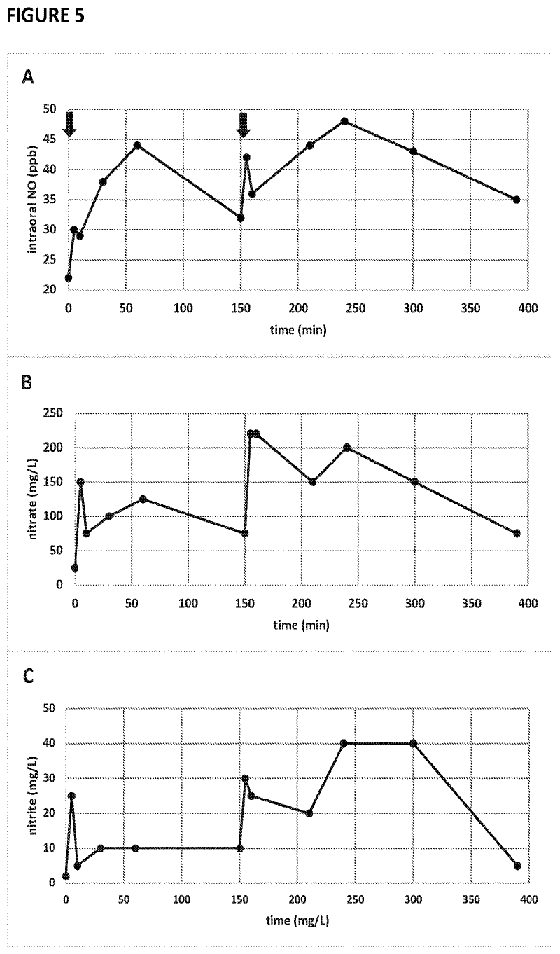

Example 4 (FIG. 5) shows intraoral nitric oxide gas formation (A), saliva nitrate (B), and saliva nitrite (C) at various times after chewing one (1) potassium nitrate-formulated gum at intermittent time points. In example 4, gum is administered at 0 time for 5 minutes and 150 min later and in both cases discarded after 5 min of chewing. ARROW(S) represents the time point of administering chewing gum for 5 min and the number of ARROW(S) indicates the number of pieces.

Example 4 is the best example of an immediate local increase in nitric oxide which in turn contributed in an additive fashion with the intermittent addition of another gum to increase both the local and systemic nitric oxide level. The unique and novel aspect of this example is that the duration of nitric oxide bioavailability was extended to 5 hours with a cumulative lower amount when administrated over time in comparison to a single high dose as shown in Example 3.

### Example 5

### Bioconversion of Nitrate Chewing Gum Increases Intraoral and Nasal Exhaled Nitric Oxide

Wherein the methods, experiment protocol, and measurements nitric oxide gas is described above for the plant-derived and potassium-ascorbate-zinc chewing gum.

NObreath® (NOB; Bedfont, Kent, UK) was used to measure oral nitric oxide gas and modified with a nose piece to capture exhaled air from a nostril where the opposing nostril is press closed during the 10 second exhalation.

NOB was used to measure nitric oxide gas. These devices were assessed based on the National Institute for Health and Clinical Excellence (NICE) guideline. NOB is designed to measure fractional exhaled nitric oxide (FeNO) in the human breath and a normal FeNO test is any number less than 25 parts per billion (ppb) in adults. Levels above this value have been used to assess inflammatory response in the lung where inducible nitric oxide synthase is activated in immune cells in response to an inflammatory response. NOB is used to evaluate nitric oxide gas generated locally in the mouth.

Participants were screened in advance, excluding asthmatics, individuals with respiratory infections and healthy individuals with levels about 35 ppb which was due to nitrate-rich beet or leafy green diets or excessive nasal breathing within 6-18 hour prior to testing. All included healthy subjects consistently exhaled between 10-30 ppb which was influenced with exercise and diet, especially, nitrate-rich plant-based foods and diets and excessive nasal breathing.

A total of 4-6 subjects were evaluated 2-4 time over the course of 2 weeks. The Figures are measurements from a single individual and representative of the other 4-6 subjects. Participates underwent the measurement in a sitting position, always holding the monitor upright. After inhaling to total lung capacity, participants then exhaled through the mouthpiece, ensuring that the visual indicator marker was held in the middle of the bands for proper measurement. The exhalation time was approximately 10 seconds. All participants were asked to repeat the measurement 3 times to verify the repeatability of the device. The baseline values ranging between 10-35 ppb is reflective of lung and respiratory tract sourced nitric oxide prior to chewing gum.

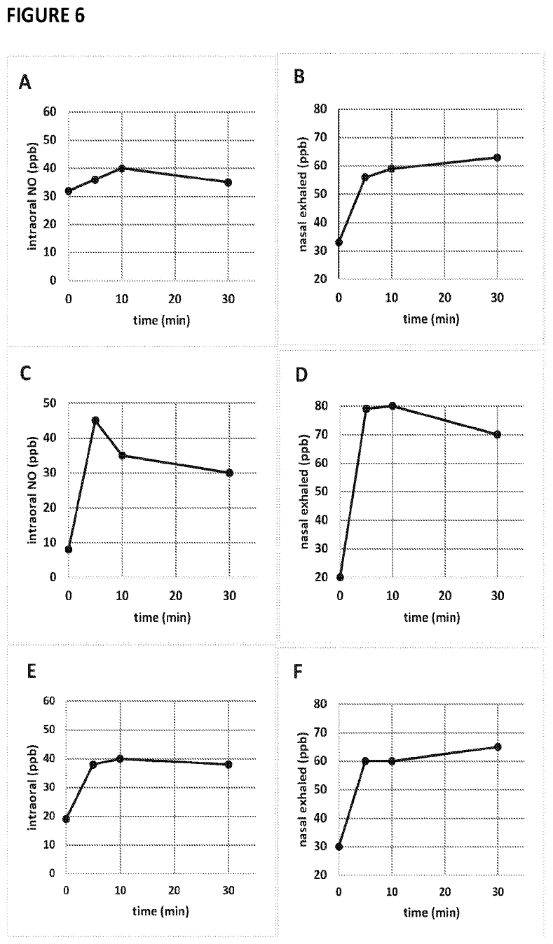

Example 5 (FIG. 6) shows intraoral (A-E) and nasal (B-F) exhalation for 10 seconds at 4 time points including 0 (pre-gum chewing) and 5, 10, 30 min after chewing one (1) piece of potassium-ascrobate-zinc gum for 5 min beginning at 0 time point.

Example 5 shows an immediate increase in both intraoral and nasal cavity nitric oxide gas formation. FIG. 6 represents 3 healthy subjects wherein oral and nasal nitric oxide measurements were taken at 4 time points; subjects 1, 2, 3 results are shown as A and B, C and D, and E and F, respectively. Baseline level of exhaled nasal nitric oxide gas was found to be 1-2× higher than oral and the magnitude of the exhaled in nitric oxide gas was found to be consistently higher in nasal compared to oral in absolute terms by 2-4× higher. Further, the ‘hang time’ of nasal nitric oxide appeared to persistent longer than that of the mouth.

Example 5 understates the importance of nitric oxide chewing gum in improving local nasal nitric oxide, especially in subject with low nasal sinus nitric oxide production, since most of the nitric oxide from the upper airway is produced in the nasal cavity. Lundberg et al (1996) reminds us recent studies have shown that inhaled NO (typically from a external device) at concentrations as low as 100 ppb significantly decrease pulmonary vascular resistance in patients with pulmonary hypertension indicating that nasally derived nitric oxide is physiologically important in the lung and, thus, act as an airborne, or “aerocrine” factor. Nitric oxide also has bacteriostatic and antiviral properties, and nasally derived nitric oxide can participate in the mucosal defense line against infections.

Hence, the invention herein is a new and unique composition and method to provide nitric oxide to the nasal cavity as exemplified in FIG. 6, thereby, supporting a source for antimicrobial nitric oxide to the nasopharynx and respiratory tract to combat both infections and manage pulmonary hypertension.

### Example 6

### Nitrate Chewing Gum Shifts Oral Microbiome to High NO Bioavailability

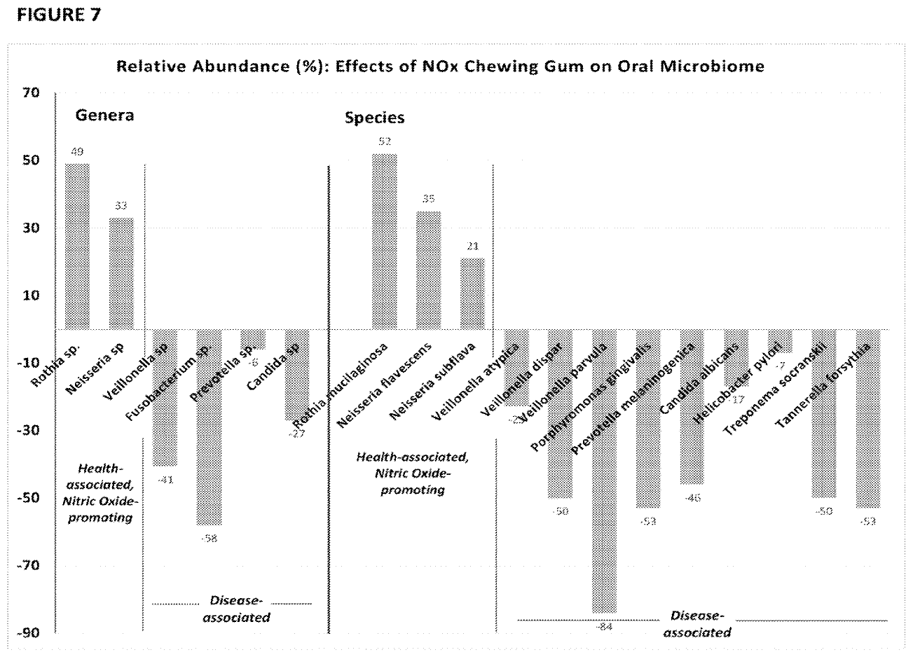

FIG. 7 shows the relative abundance of the bacterial species before and after chewing potassium nitrate-formulated gum. Whole genome metagenomic sequencing or Shotgun sequencing was used to looks at all the DNA present in a microbiome sample. Saliva samples were in subjects with low nitrite-to-nitrate ratio with Probiotic Nitric Oxide Tests by MyFitStrip® and low saliva pH (below 7) as detected with Oral Health Strips by MyFitStrips®. Saliva was collected according to the methodology provided by Bristle Oral Health Labs. Upon collection, subject chewed potassium-ascorbic acid formulated gum for for 5-10 min twice over a period 3-4 hours. At that time, nitrite-to-nitrate and pH were measure and a second saliva sample was collected for Shotgum sequencing analysis according to the procedures of Bristle Labs.

As summarized in FIG. 7, the abundance of bacterial genera capable of nitrate reduction diverged. While the abundance of Rothia and Neisseria in test group patient increased at after chewing the nitric oxide formulated chewing gum periodically for 5 min each for a total of 3 gum chewing periods over a 3 hour period, the abundance of Prevotella, Veillonlla, and Treponema among other species was reduced.

The genera Rothia and Neisseria are known for their high capacity of nitrate reduction as well as for their association with periodontal health. Especially, the Neisseria species of healthy volunteers have been shown to possess a very efficient nitrate/nitrite-reducing metabolism.

Evidence continues to show a correlation between subgingival inflammation and cardiovascular health hence a recurring link between diseases are linked. One potential mechanism is the change in the enterosalivary nitrate metabolism pathway, which affects systemically available nitric oxide directly influences cardiometabolic outcomes. For example, Treponema colonization could contribute to nitrate reduces and nitrite depletion, hence, disrupting the nitrate microflora in the mouth. Further evidence, suggest that health-associated taxa, Rothia and Neisseria were detected in healthy controls while Treponema, Porphyromonas, and Tannerella prevailed in periodontitis.

High abundances of Rothia and Neisseria and low abundances of Prevotella and Veillonella were correlated with oral microbial community related to indices of nitric oxide homeostasis and vascular health.

Nitrate-reducing bacteria Rothia and Neisseria are consistently found at higher levels in individuals free of oral disease in comparison to individuals with caries, periodontitis, and halitosis and increase when nitrate-rich diets are consumed. In contrast, bacteria normally associated with disease, such as Veillonella—which is often found with high caries—and Prevotella—associated with periodontal diseases and halitosis—decrease in the presence of nitrate-rich foods and diet. Hence, nitrate as an ecologic factor stimulating health-associated species and functions. FIG. 7 demonstrates that chewing gum can replace nitrate-rich diets and foods which works through the enterosalivary loop at relatively high ADI or concentrations of dietary nitrate whereas chewing gum delivers prebiotic nitrate directly, independent of the enterosalivary loop, to enhance health associated microbiome while reducing disease associate bacterial among other nitric oxide-sensitive infectious disease, including Helicobacter and Candida.

While the present disclosure has been discussed in terms of certain embodiments, it should be appreciated that the present disclosure is not so limited, hence, to include, restoring nitric oxide deficiency in smokers, ex-smokers, and secondhand smokers, especially among the previous smokers in their later years where nitric oxide deficiency is most pronounced with associated saliva acidity and diminished immune function. In like fashion, the composition can be modified to enhance bioactivity with the addition of specific vitamin Bs, specifically, thiamine mononitrate, nicotinamide riboside, N-acylcysteine (NAC). Furthermore, 50 mg of caffeine to synergize in the enhancement of nitric oxide-mediated activities.

The embodiments are explained herein by way of example, and there are numerous modifications, variations and other embodiments that can be employed that would still be within the scope of the present disclosure to increase NO bioavailability associated with promoting vascular health, diminishing oral bacterial and infectious agents contributing to oral-nasal disease, including, but not limited to viral and fungal infections, and restoring NO deficiencies caused by chronic conditions and exposure to irritants, including, smoking and poor dietary lifestyle.

### REFERENCES

1. Lundberg, J. O., Carlström, M. & Weitzberg, E. Metabolic effects of dietary nitrate in health and disease. Cell Metab. 28, 9-22 (2018).
2. V. Kapil, R. S., Khambata, D. A., Jones A. et al. Nitrate-Nitrite-Nitric Oxide Pathway Pharmacol. Rev. 72, 692-766 (2020).
3. Hezel, M. P. & Weitzberg, E. The oral microbiome and nitric oxide homoeostasis. Oral Dis. 21, 7-16(2015).
4. Gee, L. C., Ahluwalia, A. Dietary Nitrate Lowers Blood Pressure: Epidemiological, Pre-clinical Experimental and Clinical Trial Evidence. Curr Hypertens Rep 18, 17-(2016).
5. Doel, J J et al. Protective effect of salivary nitrate & microbial reductase against caries. Eur. J. Oral Sci. 112, 424.
6. Li et al. Salivary nitrate: an ecological factor in reducing oral acidity. Oral Microbiol Immunol. 22, 67-71 (2007).
7. Allaker, R. P., et al. Antimicrobial effect of acidified nitrite on periodontal bacteria. Oral Microbiol Immunol. 16, 253-256 (2001).
8. Sanchez, G. A. et al. Total salivary nitrates and nitrites in oral health and periodontal disease. Nitric Oxide 30, 36-31 (2014)
9. Mitsui, T. et al. Salivary nitrate may have antimicrobial effects on Desulfovibrio species. Biosci Biotechnol Biochem. 77, 2489 (2013).
10. Mazurel, D., Carda-Diéguez, M., Langenburg, T. et al. Nitrate and a nitrate-reducing Rothia aeria strain as potential prebiotic or synbiotic treatments for periodontitis. npj Biofilms Microbiomes 9, 40 (2023).
11. Rosier, B. T., Buetas, E., Moya-Gonzalvez, E. M., Artacho, A. & Mira, A. Nitrate as a potential prebiotic for the oral microbiome. Sci. Rep. 10, 12895 (2020).
12. Vanhatalo, A. et al. Nitrate-responsive oral microbiome modulates nitric oxide homeostasis and blood pressure in humans. Free Radic. Biol. Med. 124, 21-30 (2018).
13. Velmurugan S, et al. Dietary nitrate improves vascular function in patients with hypercholesterolemia: a randomized, double-blind, placebo-controlled study. Am J Clin Nutr. 103, 25-38. (2016).
14. Jockel-Schneider, Y. et al. Stimulation of the nitrate-nitrite-NO-metabolism by repeated lettuce juice consumption decreases gingival inflammation in periodontal recall patients: a randomized, double-blinded, placebo-controlled clinical trial. J. Clin. Periodontol. 43, 603-608 (2016).

15.Goh C E, et al. Association Between Nitrate-Reducing Oral Bacteria and Cardiometabolic Outcomes: Results From ORIGINS. J Am Heart Assoc. 3:e013324 (2019).

1. A nitrate-based formulation comprising:
a) chewing gum composition comprising a gum base combined with:
i. potassium nitrate and ascorbic acid; or
ii. potassium nitrate, ascorbic acid and polyphenol extract; or
iii. potassium nitrate, ascorbic acid and zinc; or
iv. potassium nitrate, ascorbic acid, zinc, and polyphenol extract;
v. plant-derived inorganic nitrate and ascorbic acid; or
vi. plant-derived inorganic nitrate, ascorbic acid, and zinc; or
vii. plant-derived inorganic nitrate, ascorbic acid, zinc, and polyphenol extract, or
b) lozenge composition comprising sorbitol, natural flavors, stevia, magnesium stearate, and natural silica or sorbitol, silicon dioxide, and citric acid combined with:
i. potassium nitrate and ascorbic acid; or
ii. potassium nitrate, ascorbic acid and polyphenol extract; or
iii. potassium nitrate, ascorbic acid and zinc; or
iv. potassium nitrate, ascorbic acid, zinc, and polyphenol extract;
v. plant-derived inorganic nitrate and ascorbic acid; or
vi. plant-derived inorganic nitrate, ascorbic acid, and zinc.

2. The chewing gum or lozenge composition of claim 1, consisting of a sugar blend, a blend of sugar alcohols, sweeteners, sorbitol, stevia, a gum base or bulk filler, flavorings, lubricants, flow agents, magnesium stearate or combinations thereof.

3. The lozenge of claim 2, wherein the lozenge comprises a slow-release formulation.

4. The chewing gum or lozenge composition of claim 2, further comprising flavorings and/or sweeteners,
wherein the flavorings comprise natural or artificial flavorings or a combination thereof;
and wherein the sweeteners comprise sugars, non-sugars, sugar alcohols, plant-derived sweeteners, including stevia, agave, coconut sugar, honey, monk fruit, raspberry and/or combinations thereof.

5. The chewing gum or lozenge composition of claim 1, wherein the potassium nitrate comprises 1-5% of the composition.

6. The chewing gum or lozenge composition of claim 5, wherein the potassium nitrate is derived from a plant source, wherein the plant source is selected from the group consisting of leafy greens, comprising celery, beet, arugula, Swiss chard, beetroot and/or combinations thereof.

7. The chewing gum or lozenge composition of claim 1, wherein ascorbic acid comprises plant-derived ascorbic acid, including but not limited to, acerola-derived ascorbic acid,
and wherein composition comprises 0.5-10, 7-15% wt/wt ascorbic acid, in a ratio of nitrate-to-ascorbic acid of 2:1 to 1:4.

8. The chewing gum or lozenge composition of claim 1, wherein the composition comprises 1-10% zinc, 4-12% L-arginine, 1-5% N-acetylcysteine, or polyphenol fruit-derived extract at 2-10 mg/dose of activated anthocyanins (per g extract at 10:1) or a combination thereof.

9. The chewing gum or lozenge composition of claim 1, wherein the composition comprises 0.5-1% zinc, 6-8% L-arginine, 3-7% N-acetylcysteine, or polyphenol fruit-derive extract at 2-10 mg/dose of activated anthocyanins (per g extract at 10:1) or a combination thereof.

10. The chewing gum or lozenge composition of claim 1, wherein the amount of nitrate per chewing gum composition is 0.15 to 2.5 mM.

11. The chewing gum or lozenge composition of claim 1,
wherein the nitric oxide level in a subject's intraoral-nasal cavity and an increase in total body bioavailability of nitric oxide is maintained by the subject by chewing one or more additional chewing gum compositions, intermittently throughout a 12-hour period for a 3-7 minute per period, per gum composition,
wherein the subject discards the gum composition after chewing it for 3-7 minutes,
and wherein the subject chews a second and third gum composition, separately, for an additional 3-7 minutes wherein each gum composition chewing period is separated by of 2-4 hours.

12. A method for enhancing nitric oxide levels in the intraoral and/or nasal cavity of a subject comprising:
the step of using of a nitrate-based formulation by the subject; and
the step of monitoring pH, nitrite, and nitrate nitric oxide levels, and comprising the use of a saliva test strip,
a) chewing gum composition comprising a gum base combined with:
i. potassium nitrate and ascorbic acid; or
ii. potassium nitrate, ascorbic acid and polyphenol extract; or
iii. potassium nitrate, ascorbic acid and zinc; or
iv. potassium nitrate, ascorbic acid, zinc, and polyphenol extract;
v. plant-derived inorganic nitrate and ascorbic acid; or
vi. plant-derived inorganic nitrate, ascorbic acid, and zinc; or
vii. plant-derived inorganic nitrate, ascorbic acid, zinc, and polyphenol extract, or
b) lozenge composition comprising sorbitol, natural flavors, stevia, magnesium stearate, and natural silica or sorbitol, silicon dioxide, and citric acid combined with:
i. potassium nitrate and ascorbic acid; or
ii. potassium nitrate, ascorbic acid and polyphenol extract; or
iii. potassium nitrate, ascorbic acid and zinc; or
iv. potassium nitrate, ascorbic acid, zinc, and polyphenol extract;
v. plant-derived inorganic nitrate and ascorbic acid; or
vi. plant-derived inorganic nitrate, ascorbic acid, and zinc.

13. The method claim 12, wherein the nitric oxide levels in a subject's intraoral-nasal cavity, and an increase in total body bioavailability of nitric oxide is maintained by the subject by chewing one or more additional chewing gum compositions, intermittently throughout a 12-hour period for a 3-7 minute per period, per gum composition,
wherein the subject discards the gum composition after chewing it for 3-7 minutes,
and wherein the subject chews a second and third gum composition, separately, for an additional 3-7 minutes wherein each gum composition chewing period is separated by of 2-4 hours.

14. The method of claim 12, wherein enhancing nitric oxide levels in the intraoral and/or nasal cavity of a subject comprises increasing the relative abundance of beneficial microflora,
wherein beneficial microflora includes but is not limited to one or more of, Rothia aeria, Rothia mucilaginosa, Neisseria flavescens, Neisseria subflava, Haemophilus parainfluenzae.

15. The method of claim 12, wherein enhancing nitric oxide levels in the intraoral and/or nasal cavity of a subject comprises decreasing the relative abundance of harmful microflora wherein harmful microflora includes but is not limited to one or more of, Tannerella forsythia, Treponema socranskii, Fusobacterium periodonticum, Porphyromonas gingivalis, Streptococcus constellatus, Fusobacterium nucleatum, Parvimonas micra, Prevotella melaninogenica, Prevotella histicola, Candida albicans, and diseases associated therewith.

16. The method of claim 12, wherein improvement of oral health comprises a reduction in dental plaque, dental cavities, tooth decay, gingivitis, halitosis, bacterial infections, fungal infections, viral infections, nasopharyngitis and associated sinus and oral infections.

17. The method of claim 12, wherein the post-fasting salivary concentration of nitrate and nitrite during chewing and post 5 minutes is at least 100 mg/L or more and 10 mg/L or more, respectively, as assessed by saliva test strips for nitrate and nitrite.

18. The method of claim 12, wherein using of a nitrate-based formulation results in an increase in saliva pH to 6.5-8.0; wherein the increase in salivary pH results in a reduction of tooth demineralization, oral acidosis, and/or acidogenic bacteria.

19. The composition of claim 1, consisting of potassium nitrate, ascorbic acid, zinc, gum base, sorbitol, maltitol, xylitol, isomaltose, natural flavors, potassium nitrate, magnesium stearate, acerola, sucralose, silicon dioxide, and zinc citrate.

20. The composition of claim 1, consisting of plant-based acerola, gum base, sorbitol, maltitol, xylitol, isomaltose, celery extract, natural flavors, magnesium stearate, acerola, sucralose or stevia, silica, polyphenols and glycerin.
